(19) United States
(12) Patent Application Publication
(10) Pub. No.: US 2005/0288387 A1
(43) Pub. Date: Dec. 29, 2005

(54) Acrylate dental compositions with improved viscosity and storage odor

(76) Inventors: Li Feng, Naperville, IL (US); Byoung I. Suh, Oak Brook, IL (US)

Correspondence Address: ICE MILLER, ONE AMERICAN SQUARE, BOX 82001, INDIANAPOLIS, IN, 46282-0200, US

(21) Appl. No.: 11/200,924

(22) Filed: Aug. 10, 2005

Related U.S. Application Data
(63) Continuation-in-part of application No. 10/224,795, filed on Aug. 21, 2002.

(51) Int. Cl. A61K 7/16

(52) U.S. Cl. 523/113

(57) ABSTRACT

Dental composition and methods of use are provided, the compositions comprising a multiacrylate compound, an initiator, and an alcohol, wherein no significant storage odor is detected after storage for a significant period of time.

This application is a continuation-in-part of U.S. patent application Ser. No. 10/224,795, filed Aug. 21, 2002, herein incorporated by reference in its entirety.

### FIELD OF THE INVENTION

The invention relates to dental composite materials, and more specifically, to dental composite materials containing multifunctional acrylate compounds and having improved viscosity and storage odor.

### BACKGROUND OF THE INVENTION

Dental sealants and adhesives are widely used in clinical settings. Desirable properties include safety, efficacy, durability, and favorable cosmetic properties. It is preferred that dental compositions be shelf stable, easy to formulate, have low viscosity to allow for easy application, and not set so rapidly as to make them difficult to apply to a patient. It is also desirable that the compositions lack unpleasant odors, particularly if the compositions are to be used in a patient's mouth.

Dental compositions frequently contain monomers that are polymerized by the dentist or technician (e.g. by light, self-cure, or dual-cure). However, many dental compositions form a problematic “oxygen inhibited layer” (OIL) or “uncured layer” on their surface. This layer's polymerization is inhibited due to the presence of molecular oxygen radicals in ambient air. As a result, incomplete polymerization occurs. Such a layer often render the surface sticky or tacky, making the dental composition more difficult to mold or shape. Such incomplete polymerization also tends to lead to lower hardness of the surface and/or no curing if a thin surface is present.

Extoral is a visible-light cured dental resin formulation sold by AFR Imaging Corp. (Portland, Oreg.). Extoral can be used for surface treatment or as a denture resin. Extoral cures rapidly and produces a glossy, hard surface upon irradiation with normal dental light. Extoral is significant in that it does not have an “oxygen inhibited layer,” even when cured with low intensity light. One difficulty with using Extoral is its volatility, leading to a very strong odor. The smell is objectionable to both patients and dentists/technicians, making it difficult to use in a laboratory, and nearly impossible to use in a clinical setting. Another difficulty is the very brittle surface created by Extoral, which may limit its use in certain dental applications such as surfaces subject to compressive forces.

Extoral's odor is suggestive of the presence of methyl methacrylate. Methyl methacrylate is a volatile compound used in several dental products. In addition to its unpleasant smell, exposure to methyl methacrylate has been linked to various health concerns. Numbness, paraesthesia, reduced pulmonary function, and reduced respiratory function have been observed in dental technicians who have been chronically exposed to methyl methacrylate (Sadoh, D. R. et al., British Dental J 186(8): 380-381, 1999; Nishiwaki, Y. et al., J Occup. Health 43: 375-378, 2001). The U.S. Environmental Protection Agency describes methyl methacrylate as an irritant of the nose and throat, and mentions that exposure for short periods of time can cause headache and fatigue (EPA 749-F-95-014 Fact Sheet, November 1994).

Efforts to overcome the unpleasant odor of methyl methacrylate while still avoiding an oxygen inhibited layer have lead to the use of formulations including multi-functional acrylate monomers. A solvent is often used to lower the viscosity of these compositions, and ethanol is a preferred solvent. However, it has been found that various dental compositions, even those without methyl methacrylate, develop an unpleasant odor during storage. It is believed that this storage odor is due to the presence of ethyl acrylate, a product of trans-esterification between the acrylate monomers and the ethanol solvent. As used herein, the term “storage odor” refers to this odor believed to be due to ethyl acrylate.

It would be of value to develop a dental composition that does not have an “oxygen inhibited layer” and that does not contain methyl methacrylate or other volatile compounds that are irritating and potentially dangerous to dentists, dental technicians, and their patients. Further, it would be desirable to have a formulation that is sold pre-mixed with a solvent, such that mixing with a solvent upon use is unnecessary or minimized. Finally, it would be of value to develop a dental composition having low viscosity that does not have a storage odor or has an odor that can be masked easily, and that remains without a storage odor for a significant shelf-life of the dental composition.

### SUMMARY OF THE INVENTION

An embodiment of the invention is directed towards dental acrylic compositions containing a multiacrylate compound and an initiator. Curing of the compositions results in surfaces lacking an oxygen inhibition layer (“OIL”). The multiacrylate compound contains at least three acrylate units per molecule.

In another embodiment, ethanol or other alcohols are used as a solvent and the presence of a storage odor is minimized by minimizing the potential catalysts for trans-esterification. This may be done by minimizing water in the composition, by neutralizing acids, by removing impurities from the acrylate monomer, and by a combination of any or all of these methods of minimizing the potential catalysts.

The formulations can further comprise other acrylate compounds, solvents, fillers, nanofillers, diluents, or other materials useful in dental formulations. The formulations are useful in applications such as dental coatings, dental sealants, and fingernail/toenail repair. When used as a dental coating or sealant, the formulations may be used on restoratives outside of the mouth or may be used in the mouth on restoratives or natural tooth material, or a combination of restoratives and natural tooth material.

Additional features of the present invention will become apparent to those skilled in the art upon consideration of the following detailed description of preferred embodiments exemplifying the best mode of carrying out the invention as presently perceived.

### DETAILED DESCRIPTION

An embodiment of the invention is directed toward dental acrylic material compositions that cure to form surfaces that lack an oxygen inhibition layer (“OIL”). The compositions preferably do not contain methyl methacrylate. The compositions preferably cure rapidly to form stable, hard, glossy surfaces. The compositions preferably lack an unpleasant odor and remain relatively odor-free after significant storage time, illustratively for 3 months to 2 years.

One embodiment of the invention is a dental composition comprising a multiacrylate compound, an initiator and a solvent. The multiacrylate compound is a chemical compound comprising at least three acrylate functionalities per molecule. The composition preferably does not contain methyl methacrylate. The composition, upon curing, preferably does not form an oxygen inhibition layer.

Presently preferred initiators are phosphine oxide photoinitiators and camphorquinone. Examples of such initiators include 2,4,6-trimethylbenzoyldiphenylphosphine oxide (TPO), TPO-L, Irgacure 819, Darocure 4265, and camphorquinone. It is expected that other initiators capable of photocleavage with or without the need for amine co-initiators will have utility according to the present invention. The initiator can generally be present at any concentration in the composition. The initiator is preferably present at a concentration that will not noticeably discolor the cured composition. Example concentration ranges of the initiator include about 1 weight percent of the composition or less, at least about 1 weight percent of the composition, at least about 2 weight percent of the composition, at least about 3 weight percent of the composition, at least about 4 weight percent of the composition, at least about 5 weight percent of the composition, at least about 6 weight percent of the composition, or at least about 7 weight percent of the composition up to saturation levels of initiator in the composition. Example concentrations of the initiator include about 3 weight percent of the composition, about 6 weight percent of the composition, and about 7 weight percent of the composition up to saturation levels of initiator in the composition. It is also expected that lower concentrations can be provided in the presence of a volatile dental solvent such that, upon evaporation, the desired higher effective initiator concentration is present in the composition.

The solvent may be any solvent known in the art, including water, butyl acetate, ethyl acetate, chloroform, acetone, ethanol, isopropanol, other lower alcohols, and mixtures thereof. Ethanol is particularly well suited as a solvent for dental compositions. Ethanol provides a low viscosity, allowing easy application of the composition, and ethanol is not as volatile as acetone, providing sufficient time for application of the product. Thus, the use of ethanol as a solvent provides a good balance between volatility and viscosity concerns. Further, ethanol has a low toxicity as compared other commonly used solvents. The solvent may be present at any concentration to achieve the desired viscosity, illustratively about 0 weight percent, 20 weight percent, about 30 weight percent, about 40 weight percent, about 50 weight percent, about 60 weight percent, about 70 weight percent, about 80 weight percent, about 90 weight percent, about 95 weight percent of the composition, and any weight percent in between these weight percents. Example concentration ranges include at least about 20 weight percent of the composition and at least about 30 weight percent of the composition, illustratively 40 to 60 weight percent of the composition. It has been found that the viscosity of multiacrylate compositions can be reduced to below 100 centipoise when the composition comprises 40 to 60 weight percent of ethanol. Optionally, the solvent may be provided free of water, illustratively in absolute form.

The multiacrylate compound can generally be any multiacrylate compound having at least three acrylate functionalities per molecule in relatively close spatial proximity to one another. Examples of such multiacrylate compounds include a hexafunctional aromatic urethane acrylate oligomer, a caprolactone modified dipentaerythritol hexaacrylate, dipentaerythritol pentaacrylate, di-trimethylolpropane tetraacrylate, trimethylolpropane triacrylate, and ethoxylated trimethylolpropane triacrylate. Other multiacrylate compounds comprising three acrylate functionalities per molecule, compounds comprising four acrylate functionalities per molecule, compounds comprising five acrylate functionalities per molecule, compounds comprising six acrylate functionalities per molecule, compounds comprising seven acrylate functionalities per molecule, compounds comprising eight acrylate functionalities per molecule, compounds comprising nine acrylate functionalities per molecule, and compounds comprising ten acrylate functionalities per molecule in relatively close spatial proximity to one another are also expected to have utility according to the present invention. Larger number of oligo-acrylates or polyacrylates could be added to the compositions.

The multiacrylate compound can generally be present at any concentration of the composition. Example concentration ranges include at least about 20 weight percent of the composition and at least about 30 weight percent of the composition. Specific concentration examples include about 20 weight percent, about 30 weight percent, about 40 weight percent, about 50 weight percent, about 60 weight percent, about 70 weight percent, about 80 weight percent, about 90 weight percent, and about 95 weight percent of the composition.

Many commercially available multiacrylate compounds contain impurities that can serve as catalysts for trans-esterification in the presence of ethanol, resulting in production of ethyl acrylate. In one embodiment, the multiacrylate compound is purified to remove the impurities. Extraction, illustratively using water or NaOH may be used to remove the impurities. Other methods of purification are within the scope of this invention.

The compositions can further comprise a co-monomer. The co-monomer preferably polymerizes with the multiacrylate compound. The co-monomer can generally be any type of co-monomer, and preferably is a non-volatile acrylate compound with a surface tension that is similar to or higher than that of the selected multifunctional acrylate compound(s) present in the composition. Presently preferred co-monomers include a monoacrylate compound, diacrylate compound, a triacrylate compound, or a tetraacrylate compound. An example monoacrylate is caprolactone acrylate. Example diacrylate compounds are tripropylene glycol diacrylate, ethoxylated bisphenol A diacrylate, polyethylene glycol diacrylate, epoxy diacrylate, urethane dimethacrylate, and urethane diacrylate. An example triacrylate compound is trimethylolpropane triacrylate. An example tetraacrylate is ditrimethylolpropane tetraacrylate. Ethoxylated forms of such acrylates may be preferred due to their relatively higher surface tension.

The compositions can further comprise fillers, nanofillers, glass particles, or other dental materials. Examples of such fillers include Ox-50, silane-treated Ox-50, glass ionomer powder IXG 1944 RGW from Ferro, which is also a fluoride release agent.

The compositions can further comprise desiccants and drying agents such as CaSO₄, alumina, silica, calcium chloride, and calcium oxide, which may need to be filtered out. Other desiccants are known in the art and may be used within the scope of this invention.

Depending on the acid value of the multifunctional acrylate compound used, the compositions can further include a base for neutralization. An amine, illustratively 2-amino-2-methyl-1,3-propanediol (AMP), may be used. Other bases, as are known in the art, may be used.

In one embodiment of the present invention where alcohol is used as the solvent, trans-esterification of the multiacrylate compound in the presence of ethanol may be minimized by the selection of the multiacrylate compound, purification of the multiacrylate compound, removal of residual water, or neutralization of acids present in the composition. A combination of any or all of these methods may be used. Illustratively, no significant storage odor is detected after storage of at least 1 day at 60° C. (equivalent to about 16-32 days at room temperature), after storage of at least 2 days at 60° C. (equivalent to about 32-64 days at room temperature), after storage of at least 3 days at 60° C. (equivalent to about 48-96 days at room temperature), after storage of at least 4 days at 60° C. (equivalent to about 64 to 128 days at room temperature), after storage of at least 5 days at 60° C. (equivalent to about 80 to 160 days at room temperature), or after storage at room temperature for at least 6 months or even 2 years.

The compositions can further comprise one or more flavorings. One illustrative flavoring is menthol, illustratively in a peppermint or spearmint flavor. Eucalyptus, wintergreen, cinnamon, and other flavorings may be used.

An additional embodiment of the invention is directed towards methods of using the above-described compositions. A method of sealing a surface can comprise obtaining a surface; applying to the surface a composition comprising a multiacrylate compound and an initiator; and curing the composition to obtain a sealed surface. The sealed surface preferably does not contain an oxygen inhibition layer and does not have an unpleasant odor, even after significant storage time.

The surface can generally be any surface to be sealed, and is presently preferred to be a dental surface, a tooth, a dental implant, an artificial tooth, a bone, a fingernail, or a toenail. Additionally, the surface may be that of a previously applied dental composition such as a dental composite. Illustratively, the sealed surface may be a dental restorative that has been sealed prior to placement in the mouth of a patient, or the sealed surface may be a dental restorative, natural tooth material, or a combination of the dental restorative and natural tooth material, and optionally may be used inside a patient's mouth.

The curing can generally be performed by any means sufficient to rapidly cure the composition. The curing step is presently preferred to comprise light curing. The light curing can be performed at low light intensity or at high light intensity. The intensity of light is preferably an intensity suitable for use in a dental laboratory or in a dentist's office. Examples of light intensity ranges include less than about 50 mW/cm², less than about 100 mW/cm², about 200 mW/cm² or less, about 300 mW/cm² or less, about 400 mW/cm² or less, about 500 mW/cm² or less, about 600 mW/cm² or less, about 800 mW/cm² or less, and about 2000 mW/cm² or less, it being understood that higher light intensities can also be employed. Specific examples of light intensities include about 50 mW/cm², about 100 mW/cm², about 150 mW/cm², about 200 mW/cm², about 250 mW/cm², about 300 mW/cm², about 350 mW/cm², about 400 mW/cm², about 450 mW/cm², about 500 mW/cm², about 600 mW/cm², about 800 mW/cm², and about 2000 mW/cm². Higher light intensities may also be used. In one embodiment, visible light is used. The visible light may be of any wavelength, illustratively in the blue range (400-500nm) or may be a mixture of various wavelengths, illustratively, white light. The intensity of light used may be chosen based on the wavelength of the light used. For example, Bisco's VIP™ Dental Light Curing system using a blue wavelength light source may be employed by the dentist. Light-curing systems for dental laboratories such as the Jeneric-Pentron Cure-Lite Plus light box system or the Triad light box system from Dentsply, Inc. may also be used for dental appliances. Bisco's NTL™ System utilizing its light source without the nitrogen environment may also be used. The time of light curing can generally be any time. Presently preferred time ranges include about two minutes or less, about one minute or less, less than about 30 seconds, less than about 20 seconds, less than about 15 seconds, less than about 10 seconds, and less than about 5 seconds. Specific examples of light curing times include about one minute, about 30 seconds, about 20 seconds, about 15 seconds, about 10 seconds, about 5 seconds, about 3 seconds, about 2 seconds, and about 1 second. Shorter light cure times are generally preferably to shorten patient time for the procedure and for the convenience of the dental practitioner.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1

### Abbreviations Used in the Examples

Table 1 lists the chemical compounds and abbreviations used throughout the Examples section.

TABLE 1

| Abbre- | | |
|---|---|---|
| viation/ | | |
| product | Chemical name | Commercial source |
| BZ | Benzophenone | Sartomer (Exton, PA) |
| CQ | Camphorquinone | Aldrich Chemical |
| CTXO | 2-Chlorotioxanthen-9-one | Aldrich Chemical |
| EDMAB | Ethyl (4-dimethylamino)benzoate | Esschem Company |
| MEHQ | Methylhydroquinone, a | Aldrich Chemical |
| | polymerization inhibitor | |
| MMA | Methyl methacrylate | Aldrich Chemical |
| OX-50 | Fumed silicon dioxide filler | Degussa |
| TMPTMA | Trimethylopropane trimethacrylate | Esschem Company |
| TPO | Lucirin TPO photoinitiator; 2,4,6- | BASF |
| | trimethylbenzoyldiphenylphosphine | (Mount Olive, NJ) |
| | oxide | |
| UDMA | Urethane dimethacrylate | Esschem |
| CD 9052 | Trifunctional acid ester | Sartomer (Exton, PA) |
| CN 120 | Epoxy diacrylate | Sartomer (Exton, PA) |
| CN 383 | Monofunctional acrylated amine | Sartomer (Exton, PA) |
| | coinitiator | |
| CN 975 | Hexafunctional aromatic urethane | Sartomer (Exton, PA) |
| | acrylate oligomer | |
| CN 983 | Urethane diacrylate | Sartomer (Exton, PA) |
| Kayarad | Caprolactone modified | Sartomer (Exton, PA) |
| DPCA20 | dipentaerythritol hexaacrylate | |
| (DP20) | | |
| Kayarad | Caprolactone modified | Sartomer (Exton, PA) |
| DPCA60 | dipentaerythritol hexaacrylate | |
| (DP60) | | |
| SR 259 | Polyethylene glycol (200) diacrylate | Sartomer (Exton, PA) |
| SR 306 | Tripropylene glycol diacrylate | Sartomer (Exton, PA) |
| SR 344 | Polyethylene glycol (400) diacrylate | Sartomer (Exton, PA) |
| SR 349 | Ethoxylated bisphenol A diacrylate | Sartomer (Exton, PA) |
| SR 350 | Trimethylolpropane trimethacrylate | Sartomer (Exton, PA) |
| SR 351 | Trimethylolpropane triacrylate | Sartomer (Exton, PA) |
| SR 355 | Ditrimethylolpropane tetraacrylate | Sartomer (Exton, PA) |
| SR 399 | Dipentaerythritol pentaacrylate | Sartomer (Exton, PA) |
| SR 459 | Caprolactone acrylate | Sartomer (Exton, PA) |
| SR 495 | Caprolactone acrylate | Sartomer (Exton, PA) |
| SR 502 | Ethoxylated trimethylolpropane | Sartomer (Exton, PA) |
| | triacrylate | |
| SR 610 | Polyethylene glycol (600) diacrylate | Sartomer (Exton, PA) |

### Example 2

### Analysis of Extoral Composition

While Extoral is a commercial product, its composition and ingredients are not publicly known. The strong objectionable odor suggested that Extoral contains methyl methacrylate. The following analytical experiments were performed in an attempt to determine the chemical composition of Extoral.

Evaporation of the volatile components of Extoral produced a viscous resin. About 40% by weight of the composition evaporated. This was assumed to be primarily methyl methacrylate.

FTIR assays of pre- and post-evaporation Extoral revealed several transitions. A peak at 1620 cm⁻¹ grows larger upon evaporation of methyl methacrylate. This peak is present in butyl acrylate, but not in butyl methacrylate. An aliphatic double-bond peak shifts from 1635.0 cm⁻¹ to 1633.8 cm⁻¹ upon evaporation, indicating a transition from a methacrylate-like character to a more acrylate-like character. The FTIR spectrum of Extoral does not suggest the presence of amines.

UV/V is spectra comparing Extoral with a TPO standard allowed estimated identification and quantification of TPO in Extoral to be about 4%.

### Example 3

### Addition of a Hexafunctional Acrylate

Compositions were prepared using CN-975, a hexafunctional aromatic urethane acrylate, MMA, and other compounds. Numbers in Table 2 represent the amount of each compound in the composition by weight percent.

TABLE 2

Composition

| Compound | AC-10 | AC-10A | AC-11 | AC-11A |
|---|---|---|---|---|
| CN 975 | 17.2 | 16.7 | 68.7 | 67.0 |
| CN 120Z | 51.5 | 50.3 | | |
| MMA | 29.5 | 28.7 | 29.5 | 28.7 |
| TPO | 1.8 | 4.3 | 1.8 | 4.3 |

Compositions AC-10 and AC-11 had sticky surfaces upon curing at 500 mW/cm² for 5 seconds using the VIP light source. FTIR showed that the surface conversion was 47.3% for AC-10 and 47.5% for AC-11, about 16% lower than for Extoral (63.2%). The higher TPO concentration in AC-10A and AC-11A enhanced the curability. The surface conversion was 65.7% for AC-10A and 58.7% for AC-11A.

The surface of cured AC-11A resembles cured Extoral: non-sticky, slick, hard, and glossy.

### Example 4

### Preparation of “No Oxygen Inhibited Layer” (NOIL) Compositions

NOIL compositions contain at least two components: a multifunctional acrylate and a photo-initiator (such as phosphine oxide). A diluent (such as ethoxylated di- or tri-acrylate) may also be added to enhance handling of the multiacrylate and/or solubility of the initiator. The compositions can contain additional materials such as solvents, polymerizable co-monomers, inhibitors, surfactants, glass filler, fluorescent or phosphorescent compounds, dyes, colorants, fluoride compounds, and other materials used in the dental and orthodontic fields.

### Example 5

### Preparation of Unfilled Resin Compositions

A mixture of multifunctional-acrylate, diluents, TPO or other initiator, and optionally MEHQ are blended together. The mixture may be heated at 60° C.-62° C. for four hours with stirring or shaking to afford a clear or hazy solution. The haziness, if any, will fade over a few days.

### Example 6

### Preparation of Filled Resin Compositions

Unfilled resin and filler such as OX 50 are combined as a slurry. The slurry is ground for 30 minutes. The grinding process often introduces air bubbles into the mixture. The bubbles can be removed by centrifugation at >1000×g for 30 minutes to afford an essentially clear solution.

### Example 7

### Illustrative monomers for Use in “No Oxygen Inhibited Layer” (NOIL) Compositions

Multifunctional acrylates and acrylated diluent co-monomers have been found to be effective ingredients in NOIL compositions. Table 3 lists exemplary compounds that have been found to be useful.

TABLE 3

| | | |
|---|---|---|
| Trade name | Chemical description | Characteristics |

Multifunctional Acrylates

| | | |
|---|---|---|
| CN 975 | Hexafunctional aromatic | Fast curing, high hardness |
| | urethane acrylate | |
| Kayarad | Acrylate of caprolactone | Fast curing, lower viscosity |
| DPCA20 | modified dipentaerythritol | |
| (DP20) | | |
| Kayarad | Acrylate of caprolactone | Fast curing, flexibility |
| DPCA60 | modified dipentaerythritol | |
| (DP60) | | |
| SR399 | Dipentaerythritol | Fast curing, high hardness |
| | pentaacrylate | |

Acrylated Co-Monomers

| | | |
|---|---|---|
| SR 344 | Polyethylene glycol (400) | Hydrophobicity, hardness |
| | diacrylate | |
| SR 349 | Ethoxylated bisphenol A | Hydrophobicity, hardness |
| | diacrylate esters | |
| SR 610 | Polyethylene glycol (600) | Low viscosity, flexibility |
| | diacrylate | |
| SR 459 | Caprolactone acrylate | Flexibility, hydrophobicity |
| CN 383 | Monofunctional acrylated | Very low viscosity, |
| | amine | polymerizing rate promoter |
| CD 9052 | Trifunctional acid ester | Adhesion promoter |

### Example 8

### Preparation of Volatile MMA Compositions Containing a Tetrafunctional Acrylate (AC-23 and -33)

A composition containing 3 g SR355 (ditrimethylolpropane tetraacrylate), 2 g MMA volatile co-monomer, 0.25 g TPO, and 3.5 mg MEHQ was prepared (“AC-23”). The composition had the characteristic strong odor of a composition containing methyl methacrylate (MMA). The composition was coated on white paper and exposed to VIP light source set at 600 mW/cm² intensity for an exposure time of 30 seconds. An oxygen inhibition layer was detected by finger touch, which revealed a soft surface even after waiting 10 minutes after exposure to the light source. These results suggest that a tetrafunctional acrylate in the presence of a volatile co-monomer MMA was insufficient to prevent formation of an oxygen inhibition layer.

An additional composition containing I g CN975, 4 g SR355 (ditrimethylolpropane tetraacrylate), 0.35 g TPO, and 2.5 mg MEHQ was prepared (“AC-33”). The composition had the characteristic strong odor of a composition containing methyl methacrylate (MMA). This composition could not be cured tack-free in less than 60 seconds exposure to the VIP light gun set at 600 mW/cm^{2 ,} and was easily scratchable even after exposure to that intensity light for 60 seconds.

### Example 9

### Preparation of a Volatile MMA Composition Containing a Pentafunctional Acrylate (AC-24)

A composition containing 3 g SR399 (dipentaerythritol pentaacrylate esters), 2 g MMA volatile co-monomer, 0.25 g TPO, and 3.5 mg MEHQ was prepared. The composition had the characteristic strong odor of a composition containing methyl methacrylate (MMA). The composition was coated on Pyramid composite, shade 3.5 (Bisco, Inc.) and cured by exposure to the VIP light gun output for 10 seconds at 300 mW/cm². No oxygen inhibition layer was detected by finger touch, suggesting that a pentafunctional acrylate in the presence of a volatile co-monomer MMA is sufficient to prevent formation of an oxygen inhibition layer.

### Example 10

### Preparation of Non-Volatile NOIL Compositions AC-15C and Filled and Colored AC-15C

A composition containing 2.7 g CN975, 3.3 g SR344, 0.333 g TPO, and 3 mg MEHQ was prepared (“AC-15C”). A filled mixture of 75% AC-15C and 25% OX50 filler by weight was also prepared. The AC-15C compositions contain a hexafunctional diacrylate (CN 975) and no MMA. No MMA odor or other appreciable odor was detected. AC-15C exhibited better light curing sensitivity than Extoral, with twice the double-bond conversion rate. AC-15 was curable scratch-free and mar free after 20 seconds exposure to 500 mW/cm² light source using VIP, or 40 seconds at 200 mW/cm² using the same curing light source. Addition of 10% methyl blue colorant in ethanol did not adversely affect the curing. The conversion rate of AC-15C in air and in a nitrogen environment was the same, indicating that it lacked an OIL layer. The Barcol hardness of AC-15C was 77 after curing 40 seconds at 200 mW/cm² exposure using the Cure-Lite Plus light box system.

### Example 11

### Preparation of Non-Volatile, NOIL Composition AC-35

A composition containing 3.22 g CN975, 1.38 g CN383, 0.4 g TPO, and 2.5 mg MEHQ was prepared. AC-35 formed a scratch-free to human fingernail and tack-free surface to touch after 10 seconds cured at 300 mW/cm² on Pyramid shade 3.5 composite using the VIP system. AC-36, prepared containing the same amounts of TPO and MEHQ but 2.76 g CN 975 and 1.84 g CN 383, was curable scratch-free to fingernail scratching and tack-free to touch using the same composite base and light cure system after 20 seconds exposure at 300 mW/cm².

### Example 12

### Preparation of Non-Volatile NOIL Compositions AC-40 and AC-40A

A composition containing 8.9 g CN975, 10.3 g SR349, 7.8 g SR610, 0.9 g CD9052, 2.1 g TPO, and 15 mg MEHQ was prepared. The composition cured into a scratch-free surface after 20 seconds exposure on Pyramid shade 3.5 composite using the VIP curing system set at 500 mW/cm². The AC-40 composition also exhibited good stability after storage under water at 60° C., curing in 20 seconds at 300 mW/cm² on Pyramid composite shade 3.5 after 30 days and 47 days, the latter being comparable to storage for 472 days at 20° C. Addition of 5% CN 383 to the composition (AC-40A) caused a scratch-free surface to form after curing at 300 mW/cm² for 15-20 seconds, or 10 seconds at 500 mW/cm² exposure on Pyramid composite using the VIP curing light system. In addition to sealing applications, this composition may also be attractive as a deep cavity filler due to its relatively high viscosity (around 1200 cps at 22° C.).

AC-40 also exhibits good compressive strength of about 282 MPa±27 MPa. Compressive strength is determined by preparing a sample in a 6 mm high by 4 mm diameter stainless steel split mold. The sample is cured at 500±50 mW/cm² for 60 seconds per side. The disc is heated at 37° C. for 15 minutes. The disc is placed in a 30 ml Nalgene bottle filled with deionized water, and heated for 23 hours at 37° C. The disc is removed, blotted dry, and cooled to room temperature for one hour. An Instron Universal Testing Instrument (model 4465) is used to determine the load reading (kg) at which the sample breaks. The compressive strength is calculated as (load reading (kg)×0.0624)/sample diameter (cm).

AC-40 passed a cytotoxicity assay performed with mouse fibroblast cells on a solid agarose surface. Toxicity, or the lack thereof, was determined by measuring the zone of lysis (if any) around the test sample after incubation at 37° C. in 5% carbon dioxide for 24 hours.

### Example 13

### Preparation of a Non-Volatile NOIL Composition DP20-5

A composition containing 5.2 g DP20, 1.5 g CN383, 2.1 g SR610, 0.5 g CD9052, 0.7 g TPO, and 4.4 mg MEHQ was prepared. The composition cured very rapidly, forming a scratch-free surface after 3-6 seconds at 500 mW/cm², 5-10 seconds at 300 mW/cm², and 60 seconds at 50-100 mW/cm² on white paper using the VIP curing light system The DP-20-5 compound also had good compressive strength (51±9 MPa as determined using the Instron test system described in the previous Example). It also exhibits a relatively lower viscosity (410 cps at 22° C.) than AC-40. This composition is attractive for use as a protective coating under and around orthodontic application on enamel, or as a margin sealant where rapid curing and flowablity are important considerations

### Example 14

### Preparation of Non-Volatile, NOIL Composition Filled DP20-5

Compositions of 10% filler, 20% filler, 30% filler, and 40% filler, and 90%, 80%, 70% and 60% DP-20-5 were made by mixing the appropriate weight amount of filler with the DP20-5 composition described in Example 13 above. The filler used was OX-50 or silanated OX-50. The sample containing 20% OX-50 filled DP20-5 was scratch free after irradiation for 5 seconds at 300 mW/cm² or for 2 seconds at 500 mW/cm². The pencil hardness was >5H. These results are similar to those obtained with the neat resin lacking filler. The filler had no noticeable effect on the curability of the resin.

### Example 15

### Preparation of Non-Volatile, NOIL Composition DP60-5

A composition containing 15 g DP60, 4.5 g SR349, 4.5 g SR459, 3 g CN383, 0.9 g CD9052, 2.1 g TPO, and 15 mg MEHQ was prepared. The composition cured to a scratch-free surface in 15 seconds at 500 mW/cm² and 20 seconds at 300 mW/cm² exposure using the VIP light gun and after coating the composition on Pyramid shade 3.5 composite. This composition can be used as a flexible coating on dental prosthetic devices, or on dentures. The composition can also be used as a narrow gap filler, where flexibility is desirable.

### Example 16

### Preparation of Non-Volatile NOIL Compositions AC-25 Containing Pentaacrylate and AC-26

A pentaacrylate and two diacrylate composition containing 3 g SR 399, 3 g SR349, 3 g SR610, 0.3 g CD9052, 0.7 g TPO, and 5 mg MEHQ was prepared (AC-25). A similar composition containing a hexaacrylate and two diacrylates was prepared by combining 3 g CN975, 3 g SR349, 3 g SR610, 0.3 g CD9052, 0.7 g TPO, and 5 mg MEHQ (AC-26). Both compositions AC-25 and AC-26 were scratch free after irradiation at 300 mW/cm² for 20 seconds, or 500 mW/cm² for 10 seconds using the VIP light gun. The pencil hardness of both materials was 1H.

### Example 17

### Evaluation of Di-, Tri-, and Tetra-Acrylates in Preparation of NOIL Surfaces

Five resins were selected to compare the ability of diacrylates, triacrylates, and tetraacrylates to prepare NOIL surfaces. Monomer resins were solubilized in acetone at a 1:1 ratio by weight. TPO initiator was added, and the acetone was allowed to evaporate in air for about 60 seconds or more. After 20 seconds evaporation, the acetone was not detectable in terms of weight loss. The compositions were irradiated using either a blue light at 500 mW/cm² or a bright white light at 200 mW/cm². The produced surfaces were evaluated as described in Table 4.

TABLE 4

| Monomer | TPO | | |
|---|---|---|---|
| (#acrylates/ | weight | Irradiation time | |
| molecule) | % | and intensity | Coating surface |
| SR-355 (4) | 5.6 | 120 seconds, 500 mW/cm² | Marrable |
| | | 120 seconds, 200 mW/cm² | Almost scratch free |
| SR-355 (4) | 7 | 120 seconds, 500 mW/cm² | Scratchable |
| | | 60 seconds, 200 mW/cm² | Scratch free |
| SR-351 (3) | 6 | 100 seconds, 500 mW/cm² | Scratch free |
| | | 40 seconds, 200 mW/cm² | Scratch free |
| SR-502 (3) | 6 | 120 seconds, 500 mW/cm² | Scratch free |
| | | 60 seconds, 200 mW/cm² | Scratch free |
| SR-350 (3) | 8 | 120 seconds, 500 mW/cm² | Thick OIL |
| | | 120 seconds, 200 mW/cm² | Thick OIL |
| SR-259 (2) | 10 | 120 seconds, 500 mW/cm² | Slightly tacky, |
| | | | thin OIL |

As used in Table 4, “Scratch free” also indicates that the surface was mar free and tack free; “Mar free” also indicates that the surface was tack free but was scratchable. The actual concentration of the TPO is double that shown in the table due to the evaporation of the acetone solvent. Resins made from tri-acrylates or tetra-acrylates could be polymerized to prepare a surface lacking an oxygen inhibition layer. One triacrylate and the diacrylate tested under these conditions were not able to produce an acceptable surface.

### Example 18

### Scratch Resistance Assay of Non-Volatile NOIL Sealants

The suitability of NOIL sealants as orthodontic sealants was evaluated using a scratch resistance assay. A surface is rubbed using pencils containing graphite of different hardnesses in an attempt to scratch the surface. The surface is evaluated by what hardness of graphite is required to create a detectable scratch.

This assay is based on ASTM D 3383-00. The sealant was brushed onto a composite disc, and was cured. The pencils were obtained from a Kimberly Graphite Drawing Kit, and had hardnesses according to the following table.

TABLE 5

| | |
|---|---|
| Softest | Hardest |

| | | | | | | | | | | | | | | |
|---|---|---|---|---|---|---|---|---|---|---|---|---|---|---|
| 8B | 7B | 6B | 5B | 4B | 3B | 2B | B | HB | F | H | 2H | 3H | 4H | 5H |

The assay further distinguishes between gouges and scratches. Scratching refers to the hardness needed to indent the surface. Gouging refers to the hardness required to scrape the adhesive from the surface. Oxygen inhibition layers often are easier to scratch than the rest of the cured adhesive, so the scratch/gouge assay is useful to detect the presence of an air inhibition layer on a surface.

The following adhesive compositions were evaluated: (DP20-5, DP-20-5 with a fluorescing agent (DP-20-5F), DP-20-5 with a fluorescing agent and 20% glass ionomer (DP-20-5-FG), Bisco One Step Adhesive System, Filled L/C Sealant, and L/C Sealant. The adhesive compositions were brushed on composite disks made from Bisco Renew Shade A2 Translucent and cured for 2, 5, 10, 20, 30, and 40 seconds using the VIP curing light system before evaluating. The first value in Table 6 is for gouging, the second value is for scratching.

TABLE 6

| Product | 2 sec | 5 sec | 10 sec | 20 sec | 30 sec | 40 sec |
|---|---|---|---|---|---|---|
| DP-20-5 | >5H/>5H | >5H/>5H | >5H/>5H | >5H/>5H | >5H/>5H | >5H/>5H |
| DP-20-5F | >5H/>5H | >5H/>5H | >5H/>5H | >5H/>5H | >5H/>5H | >5H/>5H |
| DP-20-5FG | >5H/>5H | >5H/>5H | >5H/>5H | >5H/>5H | >5H/>5H | >5H/>5H |
| One Step | <8B/<8B | >5H/<8B | >5H/<8B | >5H/<8B | >5H/<8B | >5H/<8B |
| Filled L/C Sealant | <8B/<8B | <8B/<8B | >5H/<8B | >5H/<8B | >5H/<8B | >5H/<8B |
| L/C Sealant | <8B/<8B | HB/<8B | 5H/<8B | >5H/<8B | >5H/<8B | >5H/<8B |

Conventional adhesives had significant oxygen inhibition at the surface since the softest pencil used (8B) was able to scratch it. The NOIL DP-20-5 compositions did not have oxygen inhibition layers since even the hardest pencil used (5H) had difficulty scratching the surface. The addition of a fluorescing agent and glass ionomer filler did not detrimentally affect the hardness of the DP-20-5 compositions. These NOIL compositions light cured very quickly, as evidenced by their consistent assay results across the range of time periods tested.

### Example 19

### Evaluation of Photoinitiators for the Preparation of a NOIL Surface

Six photoinitiators were individually added to a common resin containing 50 g CN-795 (hexafunctional acrylate), 50 g acetone (volatile solvent), 0.1 g Flourad FC-431 (surfactant for leveling), and 0.02 g MEHQ (inhibitor for storage). The following Table lists the initiators.

TABLE 7

| Initiator | Chemical name | Absorbance × 10⁻³ |
|---|---|---|
| Lucirin TPO (TPO) | 2,4,6-trimethylbenzoyl- | 6 |
| | diphenylphosphinate | |
| Lucirin TPO-L | ethyl-2,4,6-trimethylbenzoyl- | 4 |
| (TPOL) | phenylphosphinate | |
| Irgacure 819 (819) | bis(2,4,6-trimethylbenzoyl)- | 23 |
| | phenylphosphineoxide | |
| Camphorquinone | camphorquinone | 11 |
| (CQ) | | |
| H-Nu 470 (HNu) | 5,7-diiodo-3-butoxy-6- | 2960 |
| | fluorone | |
| PAQ | phenanthrenequinone | 466 |

The absorptivity is the absorbance in the range of 400-500 nm in acetonitrile normalized by mass. The higher the value, the higher the absorption in the visible light range. The first three initiators undergo unimolecular bond cleavage upon irradiation. The last three initiators require a co-initiator, such as an amine, to undergo a bimolecular reaction. It is understood that the choice of initiator is selected based on the desired wavelength of the light used to initiate the cure, and that any of the initiators may be used with any of the acrylate monomers discussed herein, to provide desired characteristics. The initiators (and EDMAB amine if required) were added to the resin. Samples were coated on a white mixing pad, evaporated for greater than 60 seconds, and irradiated at 500 mW/cm² using a Bisco VIP light curing system. Samples were irradiated for 5 seconds, 10 seconds, 30 seconds, and 10 seconds in the absence of air. The “no air” cure was performed between two Mylar slips, each 0.25 mm thick. The actual concentration of initiator in the resin was double that shown in the table due to the evaporation of the acetone. The surface was evaluated for percent degree of conversion as indicated in Table 8.

TABLE 8

| | | | DC, % | | | |
|---|---|---|---|---|---|---|
| | | | | | | 10 sec |
| Initiator | Initiator, % | EDMAB % | 5 sec | 10 sec | 30 sec | (no air) |
| CQ | 1 | 1 | 24 | 27 | 30 | 47 |
| CQ | 3 | 3 | 43 | 50 | 58 | 65 |
| TPO | 2 | 0 | 30 | 39 | 39 | 64 |
| TPO | 3 | 0 | 29 | 52 | 52 | 53 |
| 819 | 1.5 | 0 | 44 | 47 | 47 | 57 |
| 819 | 3 | 0 | 49 | 55 | 58 | 65 |
| TPOL | 3 | 0 | 29 | 38 | 57 | 56 |
| HNu | 0.2 | 1 | 0 | 0 | 0 | 23 |
| PAQ | 0.2 | 1 | 0 | 0 | 0 | 33 |

To confirm the observation that the presence of air leads to lower DC values, triplicate samples of resin containing TPO at 3% were irradiated in the presence and absence of air. Average DC values, and the standard deviation were determined as shown Table 9.

TABLE 9

| Irradiation time | Air | DC (std. dev.) |
|---|---|---|
| 10 seconds | Present | 46.9% (3.0%) |
| 30 seconds | Present | 51.6% (4.3%) |
| 10 seconds | Absent | 57.4% (1.3%) |

Resins were prepared and coated on composite disks. After evaporation of the acetone for greater than 60 seconds, the coatings were irradiated for 30 seconds at 300 mW/cm² white light. The pencil hardness was measured, as shown in Table 10. All surfaces were found to be tack and fingernail scratch free.

TABLE 10

| Initiator | Initiator, % | EDMAB % | Pencil hardness |
|---|---|---|---|
| CQ | 1 | 1 | 1H |
| CQ | 3 | 3 | >5H |
| TPO | 2 | 0 | 4H |
| TPO | 3 | 0 | 5H |
| 819 | 1.5 | 0 | 4H |
| 819 | 3 | 0 | >5H |
| TPOL | 3 | 0 | 4H |

These results show that initiators other than phosphine oxide, such as camphorquinone, can be used to successfully prepare a NOIL surface.

### Example 20

### Ortho Shear Bond Strength Assay

DP-20-5 NOIL compositions and L/C Bonding resins were evaluated for their shear strength. NOIL DP20-5 was prepared with and without 0.75 Lumilux Blue (a fluorescent additive) and 20% glass lonomer (X1B44RWG). Bond strength was compared against unfilled and filled L/C Bonding resins.

Human tooth enamel was etched with 37% phosphoric acid semi-gel for 15 seconds. The etchant was washed away with a jet of water, and dried thoroughly with a stream of air. One coat of the aforementioned compositions was applied as a sealant over the etched area by brushing onto the etched enamel surface, and was light cured for 10 seconds at 500 mW/cm² intensity using the Bisco VIP curing light system. An orthodontic bracket was bonded to the sealed area using bracket adhesive (Lightbond or Phase II) and cured. Bonded samples were placed in a 37° C. deionized water bath for two hours before testing. The shear test results are shown in Table 11.

TABLE 11

| Bracket | | Filled L/C | Unfilled | Filled DP-20-5 |
|---|---|---|---|---|
| adhesive | L/C Sealant | Sealant | DP-20-5 | with Flourescer |
| Lightbond | 22.85 ± 2.0 MPa | 23.04 ± 6.4 MPa | 23.08 ± 1.7 MPa | 20.96 ± 1.3 MPa |
| Phase II | 23.23 ± 5.7 MPa | 25.02 ± 5.9 MPa | 20.27 ± 2.8 MPa | 21.12 ± 1.8 MPa |

Bond strengths of the tested samples were fairly similar. These results indicate that unfilled and filled NOIL compositions display shear bond strength values suitable for use in orthodontic applications.

### Example 21

### Fluoride Release Assay

The fluoride release properties of NOIL compositions were compared against L/C sealants. The NOIL sample was DP20-5 containing 20% glass ionomer for fluoride release and 0.75% Lumilux blue for phosphorescence.

Shade disks of DP-20-5 and LC Sealant were prepared as follows. A round stainless steel mold held between polyethylene sheets and glass slabs were used to prepare discs of a fixed size. The discs were cured using two light guns set at 500±50 mW/cm². A single light gun was positioned over the center of the disc, and irradiated the disc for 20 seconds. Next, the two light guns were positioned at opposing ends of the circle (across the diameter), and irradiated the disc for 10 seconds. The two light guns were moved around the circle to irradiate the disc for a total of four times of 10 seconds (position of the two light guns around the circular discs: 0 and 180 degrees, then 90 and 270 degrees, then 135 and 315 degrees, and finally 45 and 225 degrees), in addition to the 20 second irradiation in the center). The top glass plate was removed, and the curing process repeated. The specimen was heated at 37±3° C. for 15±1 minutes. A diamond burr was used to drill a hole near the edge of the disks. The disk was sonicated in acetone to remove any air inhibited layer that may be present. The diameter and height of the disk was measured with a caliper, and the mass of the disk was determined using an analytical balance. A wire was threaded through the hole and twisted/turned such that the disk can be propped upright within a vial. An aqueous solution of sodium chloride (0.2M, 10 ml) was added to each vial, the vials were capped, and placed at 37° C. for 1 hour. Fluoride release was determined using a fluoride sensitive electrode immersed in a 50/50 TISAB/sample mixture. Three tests of each sample were performed. The results are shown in Table 12.

TABLE 12

| Sample | Fluoride release (μg/cm²) |
|---|---|
| L/C Sealant test #1 | 0.178 |
| L/C Sealant test #2 | 0.172 |
| L/C Sealant test #3 | 0.153 |
| DP-20-5 test #1 | 0.286 |
| DP-20-5 test #2 | 0.262 |
| DP-20-5 test #3 | 0.280 |

These results show that the NOIL sealants such as DP-20-5 with fluoride glass can release significantly more fluoride than the conventional L/C sealant. NOIL sealants can therefore be used in applications where fluoride release is desirable or required.

### Example 22

### Use of NOIL as a Top Surface Over a Composite

A composite can be placed appropriately in a dental restoration procedure according to the manufacturer's instructions. The final layer of composite is placed and adapted to the cavosurface margin, shaped, and contoured to the desired final form. A thin coating of a NOIL composition such as AC-40 or DP-20-5 is gently applied using a soft brush over the surface of the composite and the surrounding enamel. The NOIL-coated surface is then light cured using an appropriate light source and time of irradiation (e.g. visible blue light from a VIP light gun at 600 mW/sec² for 20-40 seconds or less). The produced surface will have a smooth, glossy surface. Further, such surface can be obtained without the need for shaping the composite with burs or abrasives as required in conventional composite applications, thereby avoiding potential damage to surrounding tooth tissue structure while also further minimizing patient time in the treatment room.

### Example 23

### Use of NOIL Over a Low Viscosity Composite

A low viscosity or flowable composite can be placed appropriately in a dental restoration procedure. The final layer of composite is placed and adapted to the cavosurface margin, shaped, and contoured to the desired final form. The composite is light cured for a short period of time such as 5 seconds to eliminate flow of the composite material. A thin coating of NOIL (such as DP-20-5) is gently applied using a soft brush over the surface of the composite and the surrounding enamel. The surface is then light cured using an appropriate light source and time of irradiation (e.g. visible blue light at 600 mW/sec²). The produced surface will have a smooth, glossy surface.

### Example 24

### Use of NOIL as a Root Surface Coating

A root surface requiring desensitization can be isolated, and scrubbed with a pumice and cavity cleanser slurry using a cotton pellet, foam pellet, or microbrush. The root surface can additionally be etched with phosphoric acid for 15 seconds if desired. A thin coating of NOIL (such as DP-20-5) or a filled sample for non-slumping purposes is gently applied using a soft brush over the root surface. The NOIL surface is thinned with a gentle stream of air, and light cured using an appropriate light source and time of irradiation (e.g. for 10 seconds with visible blue light at 600 mW/sec² using the VIP curing light system)

### Example 25

### Use of NOIL on Dental Appliances

Shear bond strength (SBS) of NOIL formulas AC-40 and DP20-5 were tested and compared to that of Fortify® Plus (Bisco, Inc., Schaumburg, Ill.). The following substrates were tested: enamel, dentin, composite, uncured composite, Rex III, porcelain, amalgam, acrylic, and Itself. The following techniques were used.

Enamel—Enamel was pumiced, rinsed and dried. Enamel was etched with 37% phosphoric acid semi-gel for 15 seconds before rinsing and drying. One coat of Fortify® Plus, AC-40 or DP20-5 was applied and light cured for 20 seconds, 20 seconds and 5 seconds respectively at 500 mW/cm². Post was bonded and sheared after being stored in 37° C. water for two hours.

Dentin—Dentin was polished on moistened 600 grit sanding paper for 30 seconds, rinsed and dried. Dentin was etched for 15 seconds using 32% phosphoric acid semi-gel. Dentin was rinsed and kept moist. 5-7 coats of All Bond 2 Primer A&B mixture were applied to moist dentin. Primed surface was lightly air-dried. One coat of Fortify® Plus, AC-40 or DP20-5 was applied and light cured for 20 seconds at 500 mW/cm². Post was bonded and sheared after being stored in 37° C. water for two hours.

Amalgam and Rex 111—Amalgam and Rex III were polished on moistened 600 grit sanding paper for 30 seconds, rinsed and dried. Surface was microetched using the Accuprep to achieve a uniform surface. One coat of Fortify Plus, AC-40 or DP20-5 was applied and light cured for 20 seconds, 20 seconds and 5 seconds respectively at 500 mW/cm². Post was bonded and sheared after being stored in 37° C. water for two hours.

Composite and Acrylic—Composite and acrylic were polished on moistened 600 grit sanding paper for 30 seconds, rinsed and dried. Surface was microetched using the Accuprep to achieve a uniform surface. Surface was etched with 32% phosphoric acid semi-gel for 15 seconds before rinsing and drying. One coat of Fortify® Plus, AC-40 or DP20-5 was applied and light cured for 20s, 20s, and 5s respectively at 500 mW/cm². Post was bonded and sheared after being stored in 37° C. water for two hours.

Porcelain—Porcelain was polished on moistened 600 grit sanding paper for 30 seconds, rinsed and dried. The surface was microetched using the Accuprep to achieve a uniform surface. The surface was etched with 4% hydrofluoric acid semi gel for 4 minutes, rinsed and dried. Generous amounts of porcelain primer was applied to the surface and allowed to air-dry. One coat of Fortify® Plus, AC-40 or DP20-5 was applied and light cured for 20 seconds, 20 seconds and 5 seconds respectively at 500 mW/cm². Post was bonded and sheared after being stored in 37° C. water for two hours.

Uncured composite—A preparation was done in acrylic using a high speed handpiece and burr. The preparation was etched with Accuprep and 32% phosphoric acid semi-gel. Etched surface was treated with One Step according to manufacturers instructions. Renew A2 Translucent was filled into the preparation. One coat of Fortify Plus, AC-40 or DP20-5 was applied and light cured for 40s at 500 mW/cm². Post was bonded and sheared after being stored in 37° C. water for two hours.

Itself—Substrates of cured Fortify® Plus, AC-40 and DP20-5 were set into acrylic. Composite and acrylic were polished on moistened 600 grit sanding paper for 30 seconds, rinsed and dried. Surface was microetched using the Accuprep to achieve a uniform surface. Surface was etched with 32% phosphoric acid semi-gel for 15 seconds before rinsing and drying. One coat of Fortify® Plus, AC-40 or DP20-5 was applied to their respective substrates and light cured for 20 seconds, 20 seconds, and 5 seconds respectively at 500 mW/cm². Post was bonded and sheared after being stored in 37° C. water for two hours.

The results of the shear bond strength (SBS) assays are shown in Table 13, where numerical values are in MPa. A #5 gel cap (bonding area 0.1684 cm²) was used in each of the foregoing tests along with an Instron (Model 4466) shear bond machine set to a crosshead speed of 5 mm/min, and the shear bond strength (SBS) was calculated in MPa by dividing the peak load by bonding area. The mean and standard deviations were calculated for five replications (N=5) for each test.

TABLE 13

| Substrate | DP20-5 | AC-40 | Fortify+ |
|---|---|---|---|
| Dentin | 13.81 ± 2.9 | 19.12 ± 2.7 | 14.82 ± 9.5 |
| Enamel | 21.58 ± 4.4 | 25.51 ± 3.3 | 24.25 ± 3.1 |
| Acrylic | 11.81 ± 4.5 | 17.62 ± 2.0 | 12.82 ± 1.8 |
| Rex III | 12.63 ± 1.2 | 17.80 ± 0.1 | 11.41 ± 1.9 |
| Amalgam | 11.84 ± 1.4 | 14.98 ± 2.3 | 11.47 ± 1.9 |
| Itself | 9.21 ± 3.2 | 14.97 ± 3.7 | 18.28 ± 3.3 |
| Composite | 17.37 ± 3.1 | 15.35 ± 7.7 | 16.83 ± 4.7 |
| Porcelain | 17.72 ± 1.7 | 17.58 ± 3.2 | 14.50 ± 1.2 |
| Uncured composite | 16.74 ± 3.1 | 13.69 ± 3.0 | 22.13 ± 7.5 |

In general, DP20-5 and AC-40 performed as well or better than Fortify® Plus, within experimental error.

The location of failure has been of particular interest in comparing the performance of dental products. Sample sets consisted of 5 specimens. Most failures occurred in the substrate (SUB) or at the interface of the substrate and sealant (S/S). There are a few occasions where it appears that there was failure at the sealant layer (SEAL). There is one case in which there appears to be a failure at the sealant and post interface (S/P). These observations are summarized in the Table 14.

TABLE 14

| Substrate | DP20-5 | AC-40 | Fortify+ |
|---|---|---|---|
| Dentin | 1SEAL; 4S/S | 2SUB; 2S/S; | 1SUB; 4S/S |
| | | 1SEAL | |
| Enamel | 1SUB; 4S/S | 2SUB; 3S/S | 3SUB; 2S/S |
| Acrylic | 3S/S; 2SEAL | 4S/S; 1SEAL | 5S/S |
| Rex III | 5S/S | 5S/S | 5S/S |
| Amalgam | 5S/S | 3SUB; 2S/S | 1SUB; 4S/S |
| Itself | 4SUB; 1S/S | 4SUB; 1S/S | 5SUB |
| Composite | 5SUB | 5SUB | 5SUB |
| Porcelain | 5SUB | 5SUB | 5SUB |
| Uncured composite | 5SUB | 3SUB; 1S/S; 1S/P | 5SUB |

### Example 26

### Use of NOIL in Fingernail or Toenail Repair Applications

DP-20-5 was tested as a fingernail repair composition as follows. A thin layer of DP20-5 was coated on a human fingernail with brushing until a smooth surface was obtained. The composition was exposed to 500 mW/cm² light intensity using VIP curing light system for about 5 seconds. The composition cured into a smooth shiny surface that was hard to the touch.

### Example 27

### Reduction of Storage Odor

The better curing acrylate monomers are often the most viscous, and, thus, it is often desirable to use a solvent. As discussed above, because ethanol provides a good balance between volatility and viscosity, and ethanol has relatively low toxicity, ethanol is a preferred solvent for multi-functional acrylate dental compositions. However, when a multi-functional acrylate was prepared using 2.82 g CN975, 0.18 g TPO, 1.5 mg MEHQ, 6.0 mg Lucerine TPO, and 3.0 g EtOH (99.5%), a storage odor was detectable by the next day. It is believed that the storage odor is due to ethyl acrylate, a product of trans-esterification between the acrylate monomers and ethanol.

In one embodiment, to reduce trans-esterification, amines were added to neutralize any residual acid, as shown in Table 15, thereby reducing catalysts of trans-esterification. Drierite was also added to one composition to remove residual water.

TABLE 15

| | AC-85A | AC-85B | AC-85C | AC-85D | AC-85E | AC-85F |
|---|---|---|---|---|---|---|
| CN975 | 1.88 g | 1.88 g | 1.88 g | 1.88 g | 1.88 g | 1.88 g |
| TPO | 0.12 g | 0.12 g | 0.12 g | 0.12 g | 0.12 g | 0.12 g |
| MEHQ | 1.0 mg | 1.0 mg | 1.0 mg | 1.0 mg | 1.0 mg | 1.0 mg |
| EtOH (99.5%) | 2.00 g | 2.00 g | 2.00 g | 2.00 g | 2.00 g | 2.00 g |
| Drierite (blue) | 0.50 g | | | 0.50 g | | |
| (138CaSO₄:9H₂O) | | | | | | |
| CN 383 | | 4-5 mg | | | | |
| EDMAB | | | 4-5 mg | | | |
| para-toluene amino-diethanol | | | | 6.0 mg | 6.0 mg | |
| (p-TID) | | | | | | |
| N₂CO₃ | | | | | | 5.0 mg |

The amine or Drierite was added to the EtOH. After 2 hours, the remaining components were added to the mixture and the composition was stored at room temperature. There was no discernable odor the next day. A mild storage odor developed after 24 hour storage at 60° C. in all samples. Similar results were obtained using 2-amino-2-methyl-1,3-propanediol (AMP) as the amine.

Additional compositions were prepared using different multi-functional acrylate monomers, as shown in Table 16.

TABLE 16

| | AC-86 | | AC-87 | |
|---|---|---|---|---|
| SR 399 | 1.88 | g | | |
| DP 20 | | | 1.88 | g |
| TPO | 0.12 | g | 0.12 | g |
| MEHQ | 1.0 | mg | 1.0 | mg |
| EtOH (99.5%) | 2.00 | g | 2.00 | g |

Neither AC-86 nor AC-87 developed a discernable storage odor, even after 5 days at 60° C. A slight storage odor began to develop after 6 days at 60 ° C. (equivalent to 3-6 months at room temperature) for AC-87, but not for AC-86. Thus, it has been found that certain multifunctional erythritol acrylates are less prone to trans-esterification in the presence of ethanol. Illustratively, the multifunctional erythritol acrylate has at least four acrylate functionalities per molecule. Improved results have been obtained by purifying the multifunctional acrylate prior to preparing the composition. These compositions also demonstrate good scratch resistance. AC -86 provided a scratch free surface after curing for 4-5 s at 500 mW/cm², while AC-87 provided a scratch free surface after curing for 12 s at 500 mW/cm² at 400-500 nm.

The multifunctional acrylate may be purified by water or NaOH extraction, or by other means, as are known in the art. In an illustrative example, purification of 5.00 g SR399 was performed using 14.0 g of 0.02 N NaOH (or DI water) and 2.00 ethyl acetate to decrease viscosity, for 10 min with constant shaking. The mixture was allowed to stand for 2 hr and then centrifuged. The top water layer was removed with a pipette and rinsed with DI water one time and 0.02 N NaOH three times. The resulting composition had a mild ethyl acetate odor. Several compositions were made, as shown in Table 17.

TABLE 17

| | AC-86B1 | | AC-87B2 | |
|---|---|---|---|---|
| Treated SR 399 | 2.82 | g | | |
| (H₂O extracted) | | | | |
| Treated SR 399 | | | 2.82 | g |
| (NaOH extracted) | | | | |
| TPO | 0.18 | g | 0.18 | g |
| MEHQ | 1.5 | mg | 1.5 | mg |
| EtOH (99.5%) | 3.00 | g | 3.00 | g |

Optionally, in addition to the above multiacrylate, initiator, inhibitor, and solvent, a surfactant, such as Additive 29, may be used to promote leveling. Illustratively, the surfactant comprises 0.01 to 1.0 percent by weight of the composition, for example 0.05 to 0.10 percent. Other components may be added, as are known in the art.

Both AC-86B1 and AC86B2 have very low viscosity (below 10 centipoise), both provide a scratch free surface after curing for 5 s at 500 mW/cm², and both have a slight ethyl acetate odor. The mild ethyl acetate odor may be masked by adding one or more flavors to the final composition. Illustratively, COE-Soft (GC America, Alsip, Ill.) may be used. A 1:1 ratio of final composition to COE-Soft results in a pleasing flavor.

It is understood that a more pleasing composition may be obtained by adding a flavor to the composition. The flavor may be used to mask an odor or simply to provide for a more pleasant product. Illustrative flavors include essential oils such as thymol, spearmint, peppermint, eucalyptus, wintergreen, and cinnamon. These and other flavors are known in the art and may be used in any of the compositions of the present invention.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention.

1. A dental composition comprising
a multiacrylate compound,
an initiator, and
an alcohol,
wherein no discernable storage odor is detected after storage of at least 1 day at 60° C.

2. The composition of claim 1, wherein the alcohol is ethanol.

3. The composition of claim 1, wherein the multiacrylate compound comprises at least four acrylate functionalities per molecule.

4. The composition of claim 3, wherein the multiacrylate compound comprises five acrylate functionalities per molecule.

5. The composition of claim 3, wherein the multiacrylate compound comprises six acrylate functionalities per molecule.

6. The composition of claim 3, wherein the multiacrylate compound is an erythritol acrylate.

7. The composition of claim 6, wherein the erythritol acrylate is selected from the group consisting of dipentaerythritol pentaacrylate, dipentaerythritol hexaacrylate, caprolactone modified dipentaerythritol pentaacrylate, and caprolactone modified dipentaerythritol hexaacrylate.

8. The composition of claim 1, wherein the multiacrylate compound has been purified to remove catalysts of trans-esterification.

9. The composition of claim 8, wherein the multiacrylate compound has been purified by water or NaOH extraction.

10. The composition of claim 1, further comprising a drying agent.

11. The composition of claim 1, further comprising a base to neutralize the composition.

12. The composition of claim 11, wherein the base is an amine.

13. The composition of claim 1, wherein the composition cures to form a surface lacking an oxygen inhibition layer.

14. The composition of claim 1, wherein the initiator is a photoinitiator activated by visible light.

15. The composition of claim 1, further comprising a surfactant.

16. The composition of claim 1, further comprising a flavor.

17. The composition of claim 1, wherein the multifunctional acrylate is dipentaerythritol pentaacrylate, and the initiator is 2,4,6-trimethylbenzoyldiphenylphosphine oxide.

18. The composition of claim 1, wherein no significant storage odor is detected after storage at room temperate for 3 months.

19. The composition of claim 1, wherein no significant storage odor is detected after storage at room temperate for 24 months.

20. The composition of claim 1, wherein no discernable storage odor is detected after storage of at least 5 days at 60° C.

21. The composition of claim 1, wherein the composition has a viscosity of below 100 centipoise.

22. A method of sealing a surface, the method comprising:
obtaining a surface;
applying to the surface a composition comprising a multiacrylate compound, an initiator, and an alcohol, wherein no discernable storage odor is detected after storage of at least 1 day at 60° C.; and
curing the composition to obtain a sealed surface.

23. The method of claim 22, wherein the surface is a dental surface.

24. The method of claim 22, wherein the surface is a dental prosthesis.

25. The method of claim 22, wherein the surface is a tooth.

26. The method of claim 22, wherein the surface is an artificial tooth.

27. The method of claim 22, wherein the surface is a fingernail or toenail.

28. The method of claim 22, wherein the multiacrylate compound comprises at least four acrylate functionalities per molecule.

29. The method of claim 22, wherein the alcohol is ethanol.

30. The method of claim 22, wherein the curing step comprises curing with visible light.

31. The method of claim 30, wherein the curing step comprises light curing at a light intensity of about 100 mW/cm² or less.

32. The method of claim 30, wherein the curing step comprises light curing at a light intensity of about 300 mW/cm² or less.

33. The method of claim 30, wherein the curing step comprises light curing at a light intensity of about 500 mW/cm² or less.

34. The method of claim 30, wherein the curing step comprises light curing at a light intensity of about 800 mW/cm² or less.

35. The method of claim 30, wherein the curing step comprises light curing for less than about 15 seconds.

36. The method of claim 22, wherein the sealed surface lacks an oxygen inhibited layer.
